(19) United States
(12) Patent Application Publication
(10) Pub. No.: US 2007/0134192 A1
(43) Pub. Date: Jun. 14, 2007

(54) Two-coat cosmetic product comprising an ester of dimerdilinoleic acid and of polyol(s)

(75) Inventors: Momoko Shimizu, Tokyo (JP); Emiko Tokunaga, Kawasaki-city (JP)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/634,890

(22) Filed: Dec. 7, 2006

Related U.S. Application Data
(60) Provisional application No. 60/754,285, filed on Dec. 29, 2005.

(30) Foreign Application Priority Data
Dec. 8, 2005 (FR) 05 53789

(51) Int. Cl. A61K 8/37

(52) U.S. Cl. 424/70.31

(57) ABSTRACT

The present invention relates to a cosmetic product for caring for and/or making up the skin and/or the lips, comprising at least: one first composition comprising, in a physiologically acceptable medium, at least one ester of dimerdilinoleic acid and of polyol(s) or an ester thereof, whose viscosity, measured at about 25° C., is greater than or equal to about 1500 mPa·s, and one second composition comprising a physiologically acceptable medium.

This non provisional application claims the benefit of French Application No. 05 53789 filed on Dec. 8, 2005 and U.S. Provisional Application No. 60/754,285 filed on Dec. 29, 2005.

The present invention relates to cosmetic care and/or make-up products intended to be applied to the skin and/or the lips, comprising at least one first and one second composition, the first composition comprising an ester of dimerdilinoleic acid and of polyol(s) or an ester thereof, and also to a two-coat makeup process for the human face and body.

The two compositions of the product according to the invention may be applied successively to either human facial skin, for example the lips, or human bodily skin.

Each composition may be a loose or compacted powder, a foundation, a makeup rouge, an eyeshadow, a concealer product, a blusher, a lipstick, a lip balm, a lip gloss, a lip pencil, an eye pencil, a mascara, an eyeliner, a nail varnish, a body makeup product or a skin colouring product.

There are many cosmetic compositions or products for which the properties of gloss and colour effect of the film deposited after application to the skin and/or the lips are desired. These properties generally contribute towards the desired aesthetic effect. However, these compositions generally come up against the problem of staying power over time of the aesthetic effects, and for example gloss staying power and colour staying power with respect to the various attacking factors to which these compositions may be subjected after their application.

For example, the staying power of these compositions and of their aesthetic properties, such as their gloss effect and their colour, may be more or less impaired after contact with a fabric, or after variations in temperature and/or humidity that may occur in the course of the day, or, for example, in the case of lipsticks, after a meal.

Many strategies have been envisaged to give cosmetic compositions and products good resistance properties, for example gloss and colour resistance, over time, without affecting the general aesthetic effect that they may produce.

For example, the use of “glossy” oils such as oily polymers, for instance high-viscosity polybutenes, esters of fatty acids or of fatty alcohols of high carbon number (typically greater than 16) or certain plant oils has been envisaged in order to give cosmetic compositions and products improved gloss and for example gloss staying power properties.

However, these compounds may, under certain circumstances, have the drawback of being tacky on application and over time, and may cause considerable discomfort to the user of these compositions.

The inventors have observed, unexpectedly, that the use of certain esters of dimerdilinoleic acids and of polyol(s) or an ester thereof, for preparing cosmetic products comprising at least two compositions, one of the two compositions comprising at least one of the said esters, makes it possible to give these compositions improved gloss staying power, without affecting their combination of aesthetic properties, while at the same time affording a comfortable sensation on application.

The inventors have observed that the presence in a first composition of a cosmetic product of at least one ester of dimerdilinoleic acid and of polyol(s) or an ester thereof makes it possible to apply a composition of very variable nature and to afford a glossy makeup of improved staying power, while at the same time maintaining a sensation of comfort over time.

For example, the makeup obtained may be a two-coat makeup.

A two-coat makeup may be suited to any makeup product for human skin, and for example of the face and/or the lips, the human body and mucous membranes, for instance the lips.

The second coat may form patterns and may be applied with a stencil, a pencil or any other instrument (sponge, finger, fine brush, coarse brush, feather, etc.).

This makeup may also be applied to makeup accessories, for instance spots or patches that adhere to the skin or the lips (such as beauty spots).

According to one exemplary embodiment, one subject of the present invention is a cosmetic composition for caring for and/or making up the skin and/or the lips, comprising at least:
one first composition comprising, in a physiologically acceptable medium, at least one ester of dimerdilinoleic acid and of polyol(s) or an ester, thereof, the viscosity of which, measured at about 25° C., is greater than or equal to about 1500 mPa·s, and
one second composition comprising a physiologically acceptable medium.

According to another exemplary embodiment, a subject of the present invention is a cosmetic product for caring for and/or making up the skin and/or the lips, comprising at least two different compositions and containing, respectively, at least one ester of dimerdilinoleic acid and of polyol(s) or an ester thereof, the viscosity of which, measured at 25° C., is greater than or equal to about 2000 mPa·s.

According to another exemplary embodiment, a subject of the present invention is a cosmetic product for caring for and/or making up skin and/or the lips, comprising at least two different compositions with at least one composition containing at least one ester of general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue,
OR₃ represents a hydrocarbon-based monoalcohol residue, and
n is an integer ranging from 1 to 15.

According to another exemplary embodiment, a subject of the present invention is a cosmetic composition for caring for and/or making up the skin and/or the lips comprising at least two different compositions, with at least one composition containing at least one ester of general formula (II) below: 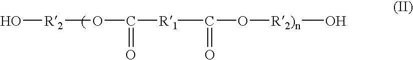
in which:
n is an integer ranging from 1 to 15,
OCR′1CO represents a dimerdilinoleate residue,
OR′2O represents a diglyceryl residue of general formula (III) below: 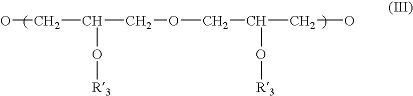
   in which:
R′₃ represents H or OR′₃ represents a fatty acid residue.

According to another exemplary embodiment, a subject of the present invention is a cosmetic product for making up and/or caring for the skin and/or the lips, comprising at least two different compositions, with at least one composition containing at least one ester of general formula (IV):
HO—R₁″—(—OCO—R₂″—COO—R₁″—)ₕ—OH (IV)
in which:
OR₁″O represents a diol dimer residue obtained by hydrogenation of a dimerdilinoleic acid,
COR₂″CO represents a hydrogenated dimerdilinoleate residue, and
h represents an integer ranging from 1 to 9, for example from 2 to 8 and for example from 4 to 6.

According to one exemplary embodiment, a subject of the present invention is a makeup kit comprising at least one product in accordance with the invention.

According to another exemplary embodiment, a subject of the present invention is the use of at least one ester of dimerdilinoleic acid and of polyol(s) or an ester thereof in accordance with the invention, for the preparation of a first composition of a cosmetic product for caring for and/or making up the skin and/or the lips, comprising at least two compositions, the said cosmetic product having improved gloss staying power.

According to one exemplary embodiment, a subject of the present invention is a process for making up and/or caring for the skin and/or the lips, comprising at least one step that consists in applying a product in accordance with the invention to at least part of a support.

According to one exemplary embodiment, the present invention makes it possible to deposit cosmetic products whose gloss, and for example whose mean gloss staying power, is improved, while at the same time not causing the user any sensation of tack or discomfort.

According to one exemplary embodiment, the present invention makes it possible to deposit cosmetic products whose colour staying power is not affected, or is even improved.

For the purposes of the present invention, the term “comfort of a cosmetic product according to the invention” is intended to denote the capacity of the said product to withstand the flaking and peeling liable to occur following movement of the skin. The comfort of a cosmetic product or composition is thus appreciated over time.

The first composition of a product according to the invention may constitute a base coat applied to the skin and/or the lips, and the second composition may constitute a top coat, or vice versa.

It is also possible to apply onto the second coat an overcoat that may or may not have the constitution of the second coat.

For the sake of simplicity, when the expression “one or more composition(s) in accordance with the invention” is used, it may denote, without reference, the first or the second composition of a product in accordance with the invention.

The subject of the present invention may be, for example, a cosmetic makeup and/or care product in the form of a foundation, a makeup rouge, an eyeshadow, a lipstick, an eyeliner, a concealer product, a body makeup product and/or a body care product.

The product according to the invention may comprise two (or a plurality of) physiologically acceptable compositions packaged separately or together in the same packaging article or in two (or a plurality of) separate or distinct packaging articles.

The compositions constituting the makeup and/or care products in accordance with the invention comprise a physiologically acceptable medium, for example a cosmetically acceptable medium, i.e. a non-toxic medium that is compatible, for example, with application to human skin and/or lips.

First Composition

A first composition may comprise, in a physiologically acceptable medium, at least one ester of dimerdilinoleic acid and of polyol(s) or an ester thereof, the viscosity of which, measured at about 25° C., is greater than or equal to about 1500 mPa·s.

The purpose of applying this first composition may be to afford the gloss effect and an improvement in the mean gloss staying power, and also improved comfort.

Thus, the surface to be made up is prepared to receive any type of cosmetic makeup and/or care composition and for example a second composition comprising a physiologically acceptable medium.

Ester of Dimerdilinoleic Acid and of Polyol(s)

In the expression “ester of dimerdilinoleic acid and of polyol(s) or an ester thereof”, the term “or an ester thereof” is intended to denote one of the derivatives of these dimerdilinoleic acid esters of polyol(s) obtained either by reaction of alcohol function(s) of the polyol, which are not employed in bonds of ester type with acid functions of the dilinoleic acid, with one or more carboxylic functions of acid molecules other than dilinoleic acid or alternatively by reaction of acid functions of the dilinoleic dimer, which are not employed in bonds of ester type with alcohol functions of the polyol, with alcohol functions of alcohol molecules other than the polyol.

Dimerdilinoleic Acid

The dimerdilinoleic acid that is suitable for use in the present invention may be obtained by polymerization reaction, for example by intermolecular dimerization of at least one linoleic acid.

The oxidation stability of the compound may be improved by hydrogenating the double bonds remaining after the dimerization reaction.

The linoleic acid dimer may also be obtained by dimerization of the hydrogenated form of linoleic acid.

The hydrogenated form of the acid or of the diacid may be partial or total, and may correspond, for example, to the saturated form, which is more oxidation-stable.

As indicated previously, the carboxylic functions of the dimerdilinoleic acid residue not engaged in the ester bond with the polyol residue (s) may be engaged in other ester bonds with other alcohol functions of alcohol molecules other than the polyol(s).

These alcohol molecules or residues may be monoalcohols or polyols.

As examples of alcohol residues that are suitable for use in the invention, mention may be made of hydrocarbon-based compounds comprising a hydroxyl function and containing from 4 to 40 carbon atoms, for example from 6 to 36 carbon atoms, for example from 8 to 32 carbon atoms, for example from 16 to 28 carbon atoms and for example from 18 to 24 carbon atoms.

As examples of monoalcohols that are suitable for the invention, mention may be made, in a non-limiting manner, of butanol, pentanol, propanol, hexanol, heptanol, octanol, decanol, dodecanol, hexadecanol, octadecanol, eicosadecanol, phytosterol, isostearol, stearol, cetol, behenol, etc.

Polyols

The term “polyol” is intended to denote any hydrocarbon-based compound comprising at least two hydroxyl functions and containing from 4 to 40 carbon atoms, for example from 6 to 36 carbon atoms, for example from 8 to 32 carbon atoms, for example from 16 to 28 carbon atoms and for example from 18 to 24 carbon atoms.

The hydrocarbon-based chains may be interrupted, where appropriate, by the presence of at least one hetero atom, and for example an oxygen atom.

A polyol or a polyol ester that is suitable for use in the present invention may comprise, for example, from 2 to 12 hydroxyl functions, in for example from 2 to 8 hydroxyl functions, and for example from 4 to 6 hydroxyl functions.

Where appropriate, the hydroxyl functions, other than those already employed in an ester bond with the dimerdilinoleic acid, may also be employed, wholly or partly with other ester bonds via reactivity with acid molecules other than the dimerdilinoleic acid.

The polyol or an ester thereof that is suitable for use in the present invention may be chosen for example from linear, branched, cyclic or polycyclic, saturated or unsaturated alcohols.

Thus, the polyol may be chosen, for example, from a diol, a triol, a tetraol, or a pentaol, or an ester thereof.

The polyol may be a diol, or an ester thereof, chosen for example from a fatty alcohol dimer, a monoglycerol or polyglycerol, a C₂₋₄ monoalkylene or polyalkylene glycol, 1,4-butanediol and pentaerythritol.

As examples of diols that are also suitable for use in the invention, mention may be made, in a non-exhaustive manner, of butanediol, pentanediol, propanediol, hexanediol, hexylene glycol, heptanediol, octanediol, nonanediol, decanediol, 1-decanediol, dodecanediol, tridecanediol, tetradecanediol, pentadecanediol, hexadecanediol, nonadecanediol, octadecanediol, cyclohexanediol, diglycerol, erythritol, pentaerythritol, xylitol, sorbitol, ethylene glycol and xylene glycol, and isomers thereof.

A fatty alcohol dimer may also be the product of hydrogenation, for example catalytic hydrogenation, of a fatty acid dimer, which is itself obtained by dimerization of at least one unsaturated fatty acid, for example of C₈ to C₃₄, for example of C₁₂ to C₂₂, for example of C₁₆ to C₂₀ and for example of C₁₈.

A fatty alcohol dimer may range from C₁₆ to C₆₈, for example from C₂₄ to C₄₄, from C₃₂ to C₄₀, or may be of C₃₆.

According to one exemplary embodiment, the diol dimer may be the product of hydrogenation of dilinoleic diacid. It may be in a saturated form.

A fatty alcohol dimer may be, for example, a dilinoleol dimer.

As an example of a diol that may be suitable for use in the invention, mention may be made for example of diglycerol.

This compound is a glycerol dimer resulting from the condensation of two molecules of glycerol, with the loss of a water molecule.

The term “diglycerol” denotes any isomer combination that can result from such a condensation, for instance linear isomers, branched isomers and, where appropriate, cyclic isomers resulting from an intramolecular dehydration of a diglycerol molecule.

The diglycerol may be obtained via any process known to those skilled in the art and for example those described in patent EP 0 750 848.

As examples of acid molecules that can interact with one or more hydroxyl functions of the polyol, not employed in the ester bond with the dimerdilinoleic acid, mention may be made, in a non-limiting manner, of molecules derived from isostearic acid, behenic acid, phytosteric acid, stearic acid or cetylic acid.

An ester that is suitable for use in the present invention may be obtained by reacting a polyol or an ester thereof with a dimerdilinoleic acid, in a molar ratio of about 1.0:0.2-1.0.

An ester that may be suitable for use in the present invention may for example be obtained by reacting a dimerdilinoleic acid with a dilinoleol and, where appropriate, at least one additional monoalcohol chosen for example from behenol, isostearol, phytosterol, stearol and cetol, and mixtures thereof.

Thus, an ester used in the context of the present invention may be used in the form of a mixture of various esters, for example.

An ester that is suitable for the invention may be obtained, for example, by reacting a glycerol, an isostearic acid and a dimerdilinoleic acid, for example, in a molar ratio of 1.0:0.2-1.0:0.5-0.9.

As examples of esters of dimerdilinoleic acid and of polyol(s) or an ester thereof suitable for the invention, mention may be made of the esters described in patent applications JP 2004-256515 and JP 2005-179377.

An ester of dilinoleic acid and of polyol(s) or an ester thereof suitable for use in the present invention may have a molecular weight ranging from about 2000 to about 25 000 g/mol, for example from about 4000 to about 20 000 g/mol, for example from about 5000 to about 20 000 g/mol, for example from about 7000 to about 15 000 g/mol and for example from about 8000 to about 10 000 g/mol.

According to an embodiment, an ester in accordance with the invention may comprise an alternating sequence of dimerdilinoleate residue(s) and of residue (s) related to the said polyol(s), and for example to the said diol(s), the said polyols or diols being, for example, as defined above.

Thus, in such a configuration, each of the two ends of the said sequence may bear, respectively, a unit OR′ and OR″ with R′ and R″ representing, independently of each other, a hydrogen atom or OR′ and OR″ representing, independently of each other, a C₂ to C₃₆, for example C₈ to C₂₄, for example C₁₂ to C₂₀ and for example C₁₆ to C₁₈ hydrocarbon-based monoalcohol residue.

According to one exemplary embodiment, R′ and R″ may both represent a hydrogen atom.

According to one exemplary embodiment, OR′ and OR″ may both represent an identical or different hydrocarbon-based monoalcohol residue.

As examples of hydrocarbon-based monoalcohol residues OR′ and OR″ that may be suitable for the invention, mention may be made of fatty alcohol residues.

According to one exemplary embodiment, an ester of dimerdilinoleic acid and of polyol(s) or an ester thereof that may be suitable for use in the present invention may have the general formula (I) below:
R₃—OCO—R₁(—COO—R₂—OCO—R₁)ₙ—COO—R₃ (I)
in which:
—COR₁CO represents a dimerdilinoleate residue,
OR₂O represents a fatty alcohol dimer residue that may range from C₁₆ to C₆₈, from C₂₄ to C₄₄, from C₃₂ to C₄₀, or may be of C₃₆,
OR₃ represents a monoalcohol residue that may range from C₄ to C₄₀, for example from C₆ to C₃₆, for example from C₈ to C₃₂, for example from C₁₆ to C₂₈, or for example from C₁₈ to C₂₄, and
n is an integer ranging from 1 to 15, for example from 2 to 10 and for example from 5 to 7.

According to one exemplary embodiment, OR₂O may represent a dimerdilinoleyl residue.

Moreover, OR₃ may represent a hydrocarbon-based monoalcohol residue chosen, for example, from behenyl, isostearyl and phytosteryl residues, and mixtures thereof.

According to another exemplary embodiment, the ester of dimerdilinoleic acid and of polyol(s) or an ester thereof that may be suitable for use in the invention may, for example, have the general formula (II) below: 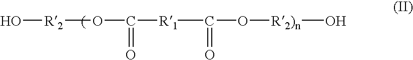
in which:
n is an integer ranging from 1 to 15, for example from 2 to 10 and for example from 5 to 7
OCR′₁CO represents a dimerdilinoleate residue,
- OR′₂O represents a diglyceryl residue of general formula (III) below: 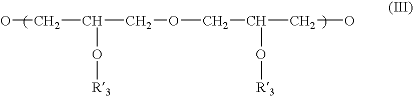
   in which:
R′₃ represents H or OR′₃ represents a fatty acid residue, that may range from C₈ to C₃₄, for example from C₁₂ to C₂₂, for example from C₁₆ to C₂₀, or for example maybe of C₁₈.

According to one exemplary embodiment, the fatty acid residue represented by OR′₃ may be an isostearyl residue.

According to one exemplary embodiment, an ester of dimerdilinoleic acid and of polyol(s) or an ester thereof, which may be suitable for use in the present invention, may be of formula (IV) below:
HO—R₁″—(—OCO—R₂″—COO—R₂″—)ₕ—OH (IV)
in which:
OR₁″O represents a diol dimer residue obtained by hydrogenation of a dimerdilinoleic acid,
COR₂″CO represents a hydrogenated dimerdilinoleate residue, and
h represents an integer ranging from 1 to 9, for example from 2 to 8 and for example from 4 to 6.

The viscosity of an ester of dimerdilinoleic acid and of polyol(s) or an ester thereof, according to the invention, may be measured according to any process known to those skilled in the art, and for example according to the conventional process described hereinbelow.

The viscosity may be measured using a cone/plate or parallel plate viscometer of Ares type (TA-Instrument) operating in kinetic sweep mode over a shear range of about 1-1000 s⁻¹ to induce a flow tension at about 1000 Pa.

The cone/plate or parallel plates may consist of a material selected from the group constituted of stainless steel, acrylic resins or polyphenylene sulfide (PPS resin).

The cone/plate diameter may be 25 mm (cone angle 0.10 radiants).

The measurement is performed at about 25° C.

Before any measurement, the stability of the sample is checked by means of the dynamic sweep period test, which makes it possible to determine if the sample is stable per se.

The shear viscosity is determined using the ETA value in the plateau region according to the flow.

The dynamic sweep period is determined at a frequency of 1.0 Hz over a period of 600 seconds.

The measurements at constant sweep rate are performed with a rate ranging from 1.0 to 1000 s⁻¹ and for example from 1.0 to 100 s⁻¹.

The viscosity of an ester of dimerdilinoleic acid and of polyol or an ester thereof suitable for use in the invention may range from about 1500 mPa·s to about 150 000 mPa·s, for example from about 2000 mPa·s to about 150 000 mpa·s, for example from about 15 000 mPa·s to about 100 000 mPa·s and for example from about 30 000 mPa·s to about 80 000 mPa·s.

An ester that is suitable for the invention may be chosen for example from the esters having the following INCI nomenclature: polyglyceryl-2 isostearate dimerdilinoleate copolymer, bis-behenyl/isostearyl/phytosteryl dimerdilinoleyl dimerdilinoleate, dimerdilinoleyl dimerdilinoleate, and mixtures thereof.

Such compounds may be obtained, for example, under the reference Hailucent ISDA (Kokyu Alcohol), Plandool-G, Lusplan DD-DA7, Lusplan PI-DA, PHY/IS-DA and Lusplan DD-DA5 (Nippon Fine Chemical Company Ltd).

According to one exemplary embodiment, ester of dimerdilinoleic acid and of polyol(s) or an ester thereof suitable for use in the invention may be present in the cosmetic compositions according to the invention in an amount sufficient to give these compositions improved cosmetic properties, for example for example in terms of gloss staying power and colour staying power.

The ester may be present in a content ranging from about 5% to about 90% by weight, for example from about 15% to about 80% by weight and for example from about 20% to about 50% by weight relative to the total weight of the composition.

Second Composition

The cosmetic makeup and/or care product according to the invention may comprise a second composition comprising a physiologically acceptable medium.

According to one exemplary embodiment, each composition of the product according to the invention may comprise, respectively, at least one ester of dimerdilinoleic acid and of polyol(s) or an ester thereof, the viscosity of which, measured at about 25° C., is greater than or equal to about 2000 mPa·s.

According to one exemplary embodiment, each composition of the product according to the invention may comprise at least one ester in accordance with the invention.

According to one exemplary embodiment, the ester(s) in accordance with the invention included in the compositions of a product according to the invention may be different.

According to one exemplary embodiment, the physiologically acceptable medium of the second composition may comprise a liquid phase that is non-volatile at room temperature and atmospheric pressure.

For the purposes of the invention, the term “non-volatile liquid phase” is intended to denote any medium capable of remaining on the skin or the lips for several hours. A non-volatile liquid phase in particular has a non-zero vapour pressure at room temperature and atmospheric pressure, of less than 0.02 mmHg (2.66 Pa) and for example less than 10⁻³ mmHg (0.13 Pa).

According to one exemplary embodiment, the first composition and the second composition may both comprise a continuous phase of the same nature.

According to one exemplary embodiment, the first or second composition, or both, may have a continuous fatty phase, for example in anhydrous form, and may contain less than 5% by weight of water, for example less than 1% by weight of water, relative to the total weight of the first or second composition.

According to one exemplary embodiment, the whole two-coat cosmetic care and/or makeup product may be in anhydrous form.

Physiologically Acceptable Medium

The compositions of a product in accordance with the invention comprise a physiologically acceptable medium.

The term “physiologically acceptable medium” is intended to denote a medium that is particularly suitable for the application of a composition according to the invention to the skin and/or the lips. The physiologically acceptable medium is generally suited to the nature of the support onto which the composition is to be applied, and to the aspect in which the composition is intended to be conditioned.

The physiologically acceptable medium may comprise an aqueous and/or water-soluble phase and/or a fatty phase.

According to one exemplary embodiment, the aqueous phase or the fatty phase may form the continuous phase of the composition.

This aqueous phase may, where appropriate, be thickened, gelled or structured by also incorporating therein a conventional aqueous gelling agent for example of mineral origin, for instance clay, and/or of organic origin, for instance an aqueous gelling polymer.

According to another exemplary embodiment, at least one of the compositions of the product in accordance with the invention may be in the form of a suspension, a dispersion, a solution, a gel or an emulsion, for example an oil-in-water (O/W) or water-in-oil (W/O) emulsion, or a multiple emulsion (W/O/W, polyol/O/W or O/W/O), or in the form of a cream, a stick, a paste, a mousse, a dispersion of vesicles, for example of ionic or nonionic lipids, a two-phase or multi-phase lotion or a powder.

For the purposes of the present invention, the emulsions contain a lipophilic phase and a hydrophilic phase, the latter phase not systematically being water.

Thus, at least one of the compositions of a product in accordance with the invention may be anhydrous.

For example, at least one of the compositions of a product according to the invention may contain, for example, a continuous fatty phase, which may contain less than 10% by weight of water, for example less than 5% by weight of water or for example less than 1% by weight of water relative to the total weight of the composition.

For example, at least one of the compositions of a cosmetic product according to the invention may be anhydrous, i.e. may contain less than 5%, for example less than 3%, for example less than 2% and for example less than 1% by weight of water relative to the total weight of the composition. These compositions may then for example be in the form of oily gels, oily liquids, pastes or sticks or alternatively in the form of a vesicular dispersion containing ionic and/or nonionic liquids.

Fatty Phase

A physiologically acceptable medium of a cosmetic composition in accordance with the present invention may comprise a fatty phase chosen for example from a liquid fatty phase, for instance oils, and a fatty substance that is solid at room temperature (20-25° C.) and atmospheric pressure, and mixtures thereof

A physiologically acceptable medium of a composition according to the invention may comprise at least one liquid fatty phase selected from the group constituted of volatile oils and non-volatile oils, and mixtures thereof.

The term “oil” means any fatty substance that is in liquid form at room temperature (20-25° C.) and at atmospheric pressure. The liquid fatty phase may also contain, in addition to oils, other compounds dissolved in the oils, such as gelling agents and/or structuring agents.

The oil(s) may be present in a proportion of from 0.1% to 99% by weight, for example from at least 1% to 90% by weight, for example from 5% to 70% by weight, for example from 10% to 60% by weight or for example from 20% to 50% by weight relative to the total weight of the cosmetic composition according to the invention.

The liquid fatty phase that may be suitable for preparing a cosmetic composition according to the invention may be selected from the group constituted of volatile or non-volatile, silicone or non-silicone oils, and mixtures thereof.

The volatile or non-volatile oils may be hydrocarbon-based oils for example of animal or plant origin, synthetic oils, silicone oils or fluoro oils, or mixtures thereof.

For the purposes of the present invention, the term “silicone oil” means an oil comprising at least one silicon atom, and for example at least one Si—O group.

The term “hydrocarbon-based oil” means an oil mainly containing hydrogen and carbon atoms, and possibly oxygen, nitrogen, sulfur and/or phosphorus atoms.

Volatile Oils

For the purposes of the present invention, the term “volatile oil” means an oil (or non-aqueous medium) capable of evaporating on contact with the skin in less than one hour, at room temperature and at atmospheric pressure. The volatile oil is a volatile cosmetic oil, which is liquid at room temperature, for example having a non-zero vapour pressure, at room temperature and atmospheric pressure, for example having a vapour pressure ranging from 0.13 Pa to 40 000 Pa (10⁻³ to 300 mmHg), preferably ranging from 1.3 Pa to 13 000 Pa (0.01 to 100 mmHg) and for example ranging from 1.3 Pa to 1300 Pa (0.01 to 10 mmHg).

The volatile hydrocarbon-based oils may be selected from the group constituted of hydrocarbon-based oils containing from 8 to 16 carbon atoms, and for example branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

Volatile oils that may also be used include volatile silicones, for instance volatile linear or cyclic silicone oils, for example those with a viscosity ≦8 centistokes (8×10⁻⁶ m²/s), and for example containing from 2 to 10 silicon atoms and for example from 2 to 7 silicon atoms, these silicones optionally comprising alkyl or alkoxy groups containing from 1 to 10 carbon atoms. As a volatile silicone oil that may be used in the invention, mention may be made for example of dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethylcyclopentane, and mixtures thereof, may also be used.

It is also possible to use a mixture of the oils mentioned above.

Non-volatile Oils

The fatty phase of a physiologically acceptable medium of a cosmetic composition according to the present invention may also comprise at least one non-volatile oil, for example an oil of high molar mass.

For the purposes of the present invention, the term “non-volatile oil” means an oil with a vapour pressure of less than 0.13 Pa and for example oils of high molar mass.

The non-volatile oils may be chosen for example from non-volatile hydrocarbon-based oils, which may be fluorinated, and/or non-volatile silicone oils.

The term “oil of high molar mass” means oils with a molar mass ranging from about 650 to about 10 000 g/mol, for example from about 750 to about 7500 g/mol, and for example ranging from about 1000 to about 5000 g/mol.

As oils of high molar mass that may be used in the present invention, mention may be made for example of oils selected from the group constituted of:
lipophilic polymers,
linear fatty acid esters with a total carbon number ranging from 35 to 70,
hydroxylated esters,
aromatic esters,
esters of C₂₄-C₂₈ branched fatty alcohols or fatty acids,
silicone oils,
oils of plant origin,
and mixtures thereof.

For example, an oil of high molar mass may be selected from the group constituted of:
a) lipophilic polymers such as:
   polybutylenes such as Indopol H-100 (of molar mass or MM=965 g/mol), Indopol H-300 (MM=1340 g/mol) and Indopol H-1500 (MM=2160 g/mol) sold or manufactured by the company Amoco,
   polyisobutylenes, for example hydrogenated polyisobutylenes, such as Panalane H-300 E sold or manufactured by the company Amoco (MM=1340 g/mol), Viseal 20000 sold or, manufactured by the company Synteal (MM=6000 g/mol) and Rewopal PIB 1000 sold or manufactured by the company Witco (MM=1000 g/mol),
   polydecenes and hydrogenated polydecenes such as: Puresyn 10 (MM=723 g/mol) and Puresyn 150 (MM=9200 g/mol) sold or manufactured by the company Mobil Chemicals,
   vinylpyrrolidone copolymers such as: the vinylpyrrolidone/1-hexadecene copolymer Antaron V-216 sold or manufactured by the company ISP (MM=7300 g/mol) and polyvinylpyrrolidone (PVP) copolymers such as the copolymers of a C₂ to C₃₀ such as a C₃ to C₂₂ alkene and combinations thereof, may be used. As examples of PVP copolymers that may be used in the invention, mention may be made of PVP/vinyl laurate, PVP/vinyl stearate, butylated polyvinylpyrrolidone, PVP/hexadecene, PVP/triacontene or PVP/acrylic acid/lauryl methacrylate copolymer,
b) esters such as:
   linear fatty acid esters with a total carbon number ranging from 35 to 70, for instance pentaerytirityl tetrapelargonate (MM=697 g/mol),
   hydroxylated esters such as polyglyceryl-2 triisostearate (MM=965 g/mol),
   aromatic esters such as tridecyl trimellitate (MM=757 g/mol),
   esters of C₂₄-C₂₈ branched fatty alcohols or fatty acids, such as those described in patent application EP-A-0 955 039 and pentaerythritol esters, and for example triisoarachidyl citrate (MM=1033.76 g/mol), pentaerythrityl tetraisononanoate (MM=697 g/mol), glyceryl triisostearate (MM=891 g/mol), glyceryl tris(2-decyl)tetradecanoate (MM=1143 g/mol), pentaerythrityl tetraisostearate (MM=1202 g/mol), polyglyceryl-2 tetraisostearate (MM=1232 g/mol) or alternatively pentaerythrityl tetrakis(2-decyl)tetradecanoate (MM=1538 g/mol),
   diol dimer esters and polyesters, such as esters of diol dimer and of fatty acid, and esters of diol dimer and of diacid, sold by the company Nippon Fine Chemical and described in the patent application FR 03 02809 filed on 6 Mar. 2003, the content of which is incorporated into the present patent application by reference,
   silicone oils such as phenyl silicones, for instance Belsil PDM 1000 from the company Wacker (MM=9000 g/mol),
   oils of plant origin such as sesame oil (MM820 g/mol),
   and mixtures thereof.

The esters of diol dimer and of monocarboxylic acid may be obtained from a monocarboxylic acid containing from 4 to 34 carbon atoms and for example from 10 to 32 carbon atoms, these acids being linear or branched, and saturated or unsaturated.

As illustrations of the monocarboxylic acids that are suitable for the invention, mention may be made for example of fatty acids.

The esters of diol dimer and of dicarboxylic acid may be obtained from a diacid dimer derived for example from the dimerization of an unsaturated fatty acid for example of C₈ to C₃₄, for example of C₁₂ to C₂₂, for example of C₁₆ to C₂₀ or for example of C₁₈.

According to one exemplary embodiment, it may for example be the diacid dimer from which the diol dimer to be esterified is also derived.

The diol dimer esters may be obtained from a diol dimer produced by catalytic hydrogenation of a diacid dimer as described above, for example hydrogenated dilinoleic diacid.

Illustrations of esters of a diol dimer that may for example be mentioned include the esters of dilinoleic diacids and of dilinoleyl diol dimers sold by the company Nippon Fine Chemical under the trade names Lusplan DD-DA5® and DD-DA7®.

An oil of high molar mass may be chosen for example from polybutylenes, polyisobutylenes, hydrogenated polyisobutylenes, polydecenes, hydrogenated polydecenes, vinylpyrrolidone copolymers such as PVP/hexadecene copolymer, pentaerythrityl tetrapelargonate, polyglyceryl-2 triisostearate, tridecyl trimellitate, triisoarachidyl citrate, pentaerythrityl tetraisononanoate, glyceryl triisostearate, glyceryl tris(2-decyl)tetradecanoate, pentaerythrityl tetraisostearate, polyglyceryl-2 tetraisostearate, pentaerythrityl tetrakis(2-decyl)tetradecanoate, phenyl silicones, sesame oil and oils of ester of acid dimer and of alcohol dimer, and mixtures thereof.

As non-volatile hydrocarbon-based oils that are also suitable for use in the invention, mention may be made for example of:
hydrocarbon-based oils of animal origin,
hydrocarbon-based oils of plant origin such as phytostearyl esters, such as phytostearyl oleate, phytostearyl isostearate and lauroyl/octyldodecyl/phytostearyl glutamate, for example sold under the name Eldew PS203 by Ajinomoto, triglycerides consisting of fatty acid esters of glycerol, the fatty acids of which may have chain lengths ranging from C₄ to C₂₄, these chains possibly being linear or branched, and saturated or unsaturated; these oils are for example heptanoic or octanoic triglycerides, wheatgerm oil, sunflower oil, grapeseed oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or alternatively caprylic/capric acid triglycerides, for instance those sold by the company Stearineries Dubois or those sold under the names Miglyol 810®, 812® and 818® by the company Dynamit Nobel,
oils of mineral or synthetic origin, for instance:
   synthetic ethers containing from 10 to 40 carbon atoms;
   linear or branched hydrocarbons of mineral or synthetic origin such as petroleum jelly, polydecenes, hydrogenated polyisobutene such as Parleam, and squalane, and mixtures thereof, and for example hydrogenated polyisobutene,
   synthetic esters, for instance oils of formula R₁COOR₂ in which R₁ represents a linear or branched fatty acid residue containing from 1 to 40 carbon atoms and R₂ represents a hydrocarbon-based chain that is for example branched, containing from 1 to 40 carbon atoms provided that R₁+R₂≧10.

The esters may be chosen for example from fatty acid esters, for example:
cetostearyl octanoate, isopropyl alcohol esters, such as isopropyl myristate or isopropyl palmitate, ethyl palnitate, 2-ethylhexyl palnitate, isopropyl stearate or isostearate, isostearyl isostearate, octyl stearate, hydroxylated esters, for instance isostearyl lactate, octyl hydroxystearate, diisopropyl adipate, heptanoates, and for example isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethylhexanoate, and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, and hydroxylated esters, for instance isostearyl lactate and diisostearyl malate;
polyol esters and pentaerythritol esters, for instance dipentaerythrityl tetrahydroxystearate/tetraisostearate;
esters of diol dimers and of diacid dimers, such as Lusplan DD-DA5® and Lusplan DD-DA7®, sold by the company Nippon Fine Chemical and described in patent application FR 03/02809 filed on March 2003;
   fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
   higher fatty acids such as oleic acid, linoleic acid or linolenic acid, and mixtures thereof; and
   dialkyl carbonates, the two alkyl chains possibly being identical or different, such as dicaprylyl carbonate sold under the name Cetiol CC® by Cognis;
   non-volatile silicone oils, for instance non-volatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenylmethyldiphenyltrisiloxanes and 2-phenylethyl trimethylsiloxysilicates, and dimethicones or phenyltrimethicones with a viscosity of less than or equal to 100 cSt, and mixtures thereof;
and mixtures thereof.

The non-volatile oils may be present in a composition according to the invention in a content ranging from 5% to 90% by weight, for example from 25% to 80% by weight and for example from 40% to 70% by weight relative to the total weight of the composition.

An oil of high molar mass that may be used in a composition according to the invention may be present in a proportion of from 5% to 40%, for example from 10% to 30% and for example from 15% to 20% of the total weight of the composition.

Solid Fatty Substances

A physiologically acceptable medium of a composition according to the invention may also comprise at least one solid fatty phase selected from the group constituted of waxes and pasty fatty substances, and mixtures thereof.

The wax is solid at room temperature (25° C.), with a reversible solid/liquid change of state, having a melting point of greater than 30° C., which may be up to 200° C., a hardness of greater than 0.5 MPa and having in the solid state an anisotropic crystal organization.

It may be a hydrocarbon-based wax, a fluoro wax and/or a silicone wax and may be of animal, plant, mineral or synthetic origin.

It may be chosen, for example, from beeswax, carnauba wax, candelilla wax, paraffin waxes, hydrogenated castor oil, synthetic waxes, for instance polyethylene waxes (preferably with a molecular weight of between 400 and 600) or Fischer-Tropsch waxes, silicone waxes, for instance alkyl or alkoxy dimethicones containing from 16 to 45 carbon atoms, ceresins or ozokerites, for instance isoparaffins with a melting point of less than 40° C., such as EMW-0003 sold by the company Nippon Seirou, α-olefn oligomers, such as the polymers Performa V® 825, 103 and 260 sold by the company New Phase Technologies; ethylene-propylene copolymers, such as Performalene® EP 700, and microcrystalline waxes with a melting point of greater than 85° C., such as Hi-Mic® 1070, 1080, 1090 and 3080, sold by Nippon Seirou, and mixtures thereof.

According to one exemplary embodiment, the wax(es) used in the cosmetic compositions in accordance with the present invention may be present in a content ranging from about 5% to about 30%, for example from about 5% to about 25%, for example from about 10% to about 20% and for example from about 10% to about 15% by weight relative to the total weight of the composition.

A physiologically acceptable medium of a cosmetic composition in accordance with the present invention may also comprise at least one pasty compound.

For the purposes of the present invention, the term “pasty compound” means a fatty compound with a reversible solid/liquid change of state, and comprising at a temperature of 23° C. a liquid fraction and a solid fraction. The term “pasty substance” also means polyvinyl laurate.

For the purposes of the invention, the pasty compound may have a hardness at 20° C. ranging from 0.001 to 0.5 MPa and for example from 0.002 to 0.4 MPa.

Among the pasty compounds that may be used in the composition according to the invention, mention may be made of lanolins and lanolin derivatives, for instance acetylated lanolins, oxypropylenated lanolins or isopropyl lanolate, and mixtures thereof. Esters of fatty acids or of fatty alcohols may also be used, for example those containing from 20 to 65 carbon atoms, for instance triisostearyl or cetyl citrate; arachidyl propionate; polyvinyl laurate; cholesterol esters, for instance triglycerides of plant origin such as hydrogenated plant oils, viscous polyesters and mixtures thereof. Triglycerides of plant origin that may be used include hydrogenated castor oil derivatives, such as Thixinr® from Rheox.

Mention may also be made of polyesters resulting from the esterification of a carboxylic acid and of an aliphatic hydroxycarboxylic acid ester. For example, Risocast® DA-L (ester derived from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3) sold by the Japanese company Kokyu Alcohol Kogyo.

As pasty compounds that may be for example suitable for formulating the cosmetic compositions in accordance with the present invention, mention may be made of hydrogenated cocoglycerides.

Mention may also be made of pasty silicone compounds such as high molecular weight polydimethylsiloxanes (PDMS) and for example those containing pendent chains of the alkyl or alkoxy type containing from 8 to 24 carbon atoms, and having a melting point of 20-55° C., for instance stearyl dimethicones, for example for example those sold by the company Dow Corning under the trade names DC2503® and DC25514®, and mixtures thereof.

Semi-Crystalline Polymer

A physiologically acceptable medium of a composition according to the invention may also comprise at least one semi-crystalline polymer.

For the purposes of the invention, the term “polymer” means a compound comprising at least 2 repeating units, for example at least 3 repeating units, and for example at least 10 repeating units.

For the purposes of the invention, the term “semi-crystalline polymer” means a polymer comprising a crystallizable portion and an amorphous portion in the backbone and having a first-order reversible temperature of phase change, for example of melting (solid-liquid transition). The crystallizable portion may be either a side chain (or pendent chain) or a block in the backbone.

When the crystallizable portion of the semi-crystalline polymer is a block of the polymer backbone, this crystallizable block is of different chemical nature from that of the amorphous blocks.

In this case, the semi-crystalline polymer may be a block copolymer, for example of the diblock, triblock or multiblock type.

When the crystallizable portion is a pendent chain on the backbone, the semi-crystalline polymer may be a homopolymer or a copolymer.

The crystallizable blocks or chains of the semi-crystalline polymers may represent at least 30% and for example at least 40% of the total weight of each polymer.

The terms “organic compound” and “of organic structure” mean a compound comprising carbon atoms and hydrogen atoms and possibly hetero atoms, for instance S, O, N or P, alone or in combination.

The semi-crystalline polymer(s) according to the invention mat have a melting point higher than the temperature of the keratin support intended to receive the said composition, for example the skin or the lips.

The melting point of a semi-crystalline polymer that is suitable for the invention may be, for example, less than about 50° C.

The melting point of a semi-crystalline polymer that is suitable for the invention may for example be greater than or equal to about 25° C. and less than about 45° C. For example, the melting point of the semi-crystalline polymer may be greater than or equal to about 35° C. and less than about 40° C.

The melting point values correspond to the melting point measured using a differential scanning calorimeter (D.S.C.) such as the calorimeter sold under the name DSC 30 by the company Mettler, with a temperature rise of 5 or 10° C. per minute. The melting point under consideration is the point corresponding to the temperature of the most endothermic peak in the thermogram.

According to the invention, the semi-crystalline polymers may be soluble in a fatty phase, for example to at least 1% by weight, at a temperature above their melting point.

The semi-crystalline polymers of the invention containing crystallizable blocks may be block or multiblock copolymers. They may be obtained via polymerization of a monomer containing reactive double bonds (or ethylenic bonds) or via polycondensation. When the polymers of the invention are polymers containing crystallizable side chains, these polymers may advantageously be in random form.

A semi-crystalline polymer that may be suitable for use in the present invention may be selected from the group constituted of a polymer comprising a) a hydrocarbon-based or silicone polymer backbone and b) at least one crystallizable organic side chain and/or a crystallizable organic block forming part of the polymer backbone of the said polymer, the said polymer having a number-average molecular mass of greater than 2000.

The semi-crystalline polymers of the invention may be of synthetic origin.

The semi-crystalline polymers that may be used in the invention may for example be selected from the group constituted of:
block copolymers of polyolefins of controlled crystallization, whose monomers are described in EP-A-0 951 897,
polycondensates, for example of aliphatic or aromatic or aliphatic/aromatic polyester type,
homopolymers or copolymers bearing at least one crystallizable side chain and homopolymers or copolymers bearing at least one crystallizable block in the backbone, for instance those described in document U.S. Pat. No. 5,156,911,
homopolymers or copolymers bearing at least one crystallizable side chain, fort example bearing fluoro group(s), such as those described in document WO-A-01/19333,
copolymers comprising carboxylate groups and polydimethylsiloxane groups,
and mixtures thereof.

A) Semi-crystalline Polymers Containing Crystallizable Side Chains

Mention may be made for example of those defined in documents U.S. Pat. No. 5,156,911 and WO-A-01/19333.

They are homopolymers or copolymers comprising from 50% to 100% by weight of units resulting from the polymerization of one or more monomers bearing a crystallizable hydrophobic side chain.

These homopolymers or copolymers may be of any nature, provided that they meet the conditions mentioned hereinbelow with, for example , the characteristic of being soluble or dispersible in the fatty phase, by heating above their melting point mp. They can result:
from the polymerization, for example the free-radical polymerization, of one or more monomers containing reactive or ethylenic double bond(s) with respect to a polymerization, namely a vinyl, (meth)acrylic or allylic group,
from the polycondensation of one or more monomers bearing co-reactive groups (carboxylic acid, sulfonic acid, alcohol, amine or isocyanate), such as, for example, polyesters, polyurethanes, polyethers, polyureas or polyamides.

In general, the crystallizable units (chains or blocks) of semi-crystalline polymers according to the invention are derived from monomer(s) containing crystallizable block(s) or chain(s), used for manufacturing semi-crystalline polymers. These polymers may be chosen for example from homopolymers and copolymers resulting from the polymerization of at least one monomer containing crystallizable chain(s) that may be represented by the formula:

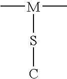

with M representing an atom of the polymer backbone,

S representing a spacer, and

C representing a crystallizable group.

The crystallizable chains “—S—C” may be aliphatic or aromatic, and optionally fluorinated or perfluorinated.

“S” for example represents a group (CH₂)ₙ or (CH₂CH₂O)ₙ or (CH₂O), which may be linear or branched or cyclic, with n being an integer ranging from 0 to 22.

“S” may be for example a linear group.

“S” and “C” may be for example different.

When the crystallizable chains are hydrocarbon-based aliphatic chains, they may comprise hydrocarbon-based alkyl chains containing at least 11 carbon atoms and not more than 40 carbon atoms and for example not more than 24 carbon atoms. They may for example be aliphatic chains or alkyl chains containing at least 12 carbon atoms, and they may be, for example C₁₄-C₂₄, for example C₁₆-C₂₂, alkyl chains. When they are fluoroalkyl or perfluoroalkyl chains, they may contain at least 11 carbon atoms, at least 6 of which carbon atoms are fluorinated.

As examples of semi-crystalline homopolymers or copolymers containing crystallizable chain(s), mention may be made of those resulting from the polymerization of one or more of the following monomers: (meth)acrylates of saturated alkyls with the alkyl group being C₁₁-C₂₄, perfluoroalkyl (meth)acrylates with a C₁₄-C₁₅ perfluoroalkyl group, N-alkyl(meth)acrylamides with the alkyl group being C₁₄ to C₂₄ with or without a fluorine atom, vinyl esters containing alkyl or perfluoroalkyl chains with the alkyl group being C₁₄ to C₂₄ (with at least 6 fluorine atoms per perfluoroalkyl chain), vinyl ethers containing alkyl or perfluoroalkyl chains with the alkyl group being C₁₄ to C₂₄ and at least 6 fluorine atoms per perfluoroalkyl chain, C₁₄ to C₂₄ α-olefins such as, for example, octadecene, para-alkylstyrenes with an alkyl group containing from 12 to 24 carbon atoms, and mixtures thereof.

When the polymers result from a polycondensation, the hydrocarbon-based and/or fluorinated crystallizable chains as defined above may be borne by a monomer that may be a diacid, a diol, a diamine or a diisocyanate.

When the polymers that are the subject of the invention are copolymers, they additionally contain from 0 to 50% of groups Y or Z resulting from the copolymerization:

α) of Y which is a polar or non-polar monomer or a mixture of the two:

When Y is a polar monomer, it is either a monomer bearing polyoxyalkylenated groups (for example oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

When Y is a non-polar monomer, it may be an ester of the linear, branched or cyclic alkyl (meth)acrylate type, a vinyl ester, an alkyl vinyl ether, an α-olefin, styrene or styrene substituted with a C₁ to C₁₀ alkyl group, for instance α-methylstyrene, or a macromonomer of the polyorganosiloxane type containing vinyl unsaturation.

For the purposes of the invention, the term “alkyl” means a saturated group for example of C₈ to C₂₄, except where otherwise mentioned.

β) of Z which is a polar monomer or a mixture ofpolar monomers.

In this case, Z has the same definition as the “polar Y” defined above.

For example, the semi-crystalline polymers containing a crystallizable side chain are alkyl (meth)acrylate or alkyl(meth)acrylamide homopolymers with an alkyl group as defined above, and for example of C₁₄-C₂₄, for example of C₁₆-C₂₀, copolymers of these monomers with a hydrophilic monomer preferably of different nature from (meth)acrylic acid, for instance N-vinylpyrrolidone or hydroxyethyl (meth)acrylate, and mixtures thereof.

The semi-crystalline polymer(s) containing a crystallizable side chain may have a weight-average molecular mass Mp ranging from 5000 to 1 000 000 g/mol, for example from 10 000 to 800 000 g/mol, for example from 15 000 to 500 000 g/mol and for example from 100 000 to 200 000 g/mol.

The semi-crystalline polymer may for example be made of the Intelimer® products from the company Landec described in the brochure “Intelimer® polymers”, Landec IP22 (Rev. 4-97). These polymers are in solid form at room temperature (25° C.). They bear crystallizable side chains and correspond to the above general formula.

For example, the Intelimer® product IPA 13-1 from the company Landec may be chosen, which is a polystearyl acrylate with a molecular weight of about 145 000 and a melting point of 49° C.

The semi-crystalline polymers may for example be those described in Examples 3, 4, 5, 7 and 9 of patent U.S. Pat. No. 5,156,911, containing a —COOH group, resulting from the copolymerization of acrylic acid and of a C₅ to C₁₆ alkyl (meth)acrylate with a melting point ranging from 20° C. to 35° C., and for example from the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of octadecyl (meth)acrylate in a 2.5/97.5 ratio.

It is also possible to use the polymer Structure “O” from National Starch, such as that described in document U.S. Pat. No. 5,736,125 with a melting point of 44° C.

The semi-crystalline polymers may for example be semi-crystalline polymers with crystallizable pendent chains comprising fluoro groups, as described in Examples 1, 4, 6, 7 and 8 of document WO-A-01/19333.

It is also possible to use the semi-crystalline polymers obtained by copolymerization of stearyl acrylate and of acrylic acid or of NVP, as described in document U.S. Pat. No. 5,519,063 or EP-A-0 550 745.

It is also possible to use the semi-crystalline polymers obtained by copolymerization of behenyl acrylate and of acrylic acid or of NVP, such as those described in documents U.S. Pat. No. 5,519,063 and EP-A-550 745.

B) Polymers Bearing at Least One Crystallizable Block in the Backbone

This may also be a case of polymers that are soluble or dispersible in the fatty phase by heating above their melting point mp. These polymers may for example be block copolymers consisting of at least two blocks of different chemical nature, one of which is crystallizable.

The polymer bearing at least one crystallizable block in the backbone may be selected from the group constituted of block copolymers of olefin or of cycloolefin containing a crystallizable chain, for instance those derived from the block polymerization of:
cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1 ,4,5,8-dimethano-1,2,3,4,4a,5,8a-octahydronaphthalene, dicyclopentadiene, or mixtures thereof,
with ethylene, propylene, 1-butene, 3-methyl-1-butene, 1-hexene, 4-methyl-1-pentene, 1-octene, 1-decene or 1-eicosene, or mixtures thereof,
and for example copoly(ethylene/norbornene) blocks and (ethylene/propylene/ethylidene-norbornene) block terpolymers.
   Those resulting from the block copolymerization of at least 2 C₂-C₁₆, and for example C₄-C₁₂, α-olefins such as those mentioned above and for example block bipolymers of ethylene and of 1-octene may also be used.

The polymer bearing at least one crystallizable block in the backbone may be selected from the group constituted of copolymers containing at least one crystallizable block, the rest of the copolymer being amorphous at room temperature. These copolymers may also contain two crystallizable blocks of different chemical nature.

According to one exemplary embodiment, the copolymers may be those that simultaneously contain, at room temperature, a crystallizable block and an amorphous block that are both hydrophobic and lipophilic, sequentially distributed. Mention may be made, for example, of polymers containing one of the crystallizable blocks and one of the amorphous blocks below:
block that is crystallizable by nature, of polyester type, for instance poly(alkylene terephthalate), or of polyolefin type, for instance polyethylenes or polypropylenes,
amorphous and lipophilic block, for instance amorphous polyolefins or copoly(olefin)s such as poly(isobutylene), hydrogenated polybutadiene or hydrogenated poly(isoprene).

As examples of such copolymers containing a crystallizable block and an amorphous block, mention may be made of:

α) poly(ε-caprolactone)-b-poly(butadiene) block copolymers, for example used hydrogenated, such as those described in the article “Melting behavior of poly(-caprolactone)-block-polybutadiene copolymers” from S. Nojima, Macromolecules, 32, 3727-3734 (1999),

β) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

γ) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30,1053-1068 (1997),

δ) the poly(ethylene)-b-poly(ethylethylene) block copolymers cited in the general article “Crystallization in block copolymers” by I. W. Hamley, Advances in Polymer Science, Vol. 148, 113-137 (1999).

C) Polycondensates of Aliphatic or Aromatic or Aliphatic/aromatic Polyester Type

The polyester polycondensates may be selected from the group constituted of aliphatic polyesters. Their molecular mass is, for example, greater than or equal to about 200 g/mol and less than or equal to about 15 000 g/mol, and for example greater than or equal to about 1000 g/mol and less than or equal to 10 000 g/mol, for example greater than or equal to about 2000 g/mol and less than or equal to about 5000 g/mol.

The polyester polycondensates may for example be selected from the group constituted of polycaprolactones. For example, the polycaprolactones may be selected from the group constituted of ε-caprolactone homopolymers. The homopolymerization may be initiated with a diol, for example a diol containing from 2 to 10 atoms, such as diethylene glycol, 1,4-butanediol or neopentyl glycol.

Polycaprolactones may be used for example, for example those sold under the name CAPA® 240 (melting point of 68° C. and molecular weight of 4000 g/mol), 223 (melting point of 48° C. and molecular weight of 2000 g/mol), 222 (melting point of 48° C. and molecular weight of 2000 g/mol), 217 (melting point of 44° C. and molecular weight of 1250 g/mol), 2125 (melting point of 45° C. and molecular weight of 1250) g/mol, 212 (melting point of 45° C. and molecular weight of 1000 g/mol), 210 (melting point of 38° C. and molecular weight of 1000 g/mol), 205 (melting point of 39° C. and molecular weight of 830 g/mol) by the company Solvay, or PCL-300 and PCL-700 by the company Union Carbide.

The CAPA® 2125 whose melting point is between 35 and 45° C. and whose weight-average molecular mass is equal to 1250 g/mol may for example be used.

D) Copolymer Comprising Carboxylate Groups and Polydimethylsiloxane Groups

In the present patent application, the expression “copolymer comprising carboxylate groups and polydimethylsiloxane groups” means a copolymer obtained from the polymerization of (a) one or more carboxylic (acid or ester) monomers, with (b) one or more polydimethylsiloxane (PDMS) chains comprising at least one polymerizable radical.

Among the “carboxylic monomers” that are suitable for use in the invention, mention may be made of carboxylic acid monomers and carboxylic acid ester monomers.

Thus, the monomer (a) may be chosen, for example, from acrylic acid, methacrylic acid, maleic acid, fumaric acid, itaconic acid, crotonic acid, esters thereof and mixtures of these monomers. Esters that may be mentioned include the following monomers methyl, ethyl, stearyl, butyl or 2-ethylhexyl: acrylate, methacrylate, maleate, fumarate, itaconate and/or crotonate, and mixtures thereof. According to one exemplary embodiment of the invention, the monomers in ester form may be selected from the group constituted of linear or branched, preferably C₁-C₂₄ and for example C₁-C₂₂ alkyl acrylates and methacrylates, the alkyl radical being selected from the group constituted of methyl, ethyl, stearyl, butyl and 2-ethylhexyl radicals, and mixtures thereof.

According to one exemplary embodiment of the invention, the copolymer may comprise as carboxylate groups at least one group selected from the group constituted of acrylic acid and methacrylic acid, and methyl, ethyl, stearyl, butyl or 2-ethylhexyl acrylate or methacrylate, and mixtures thereof.

In the present patent application, the term “polydimethylsiloxanes” (also known as organopolysiloxanes and abbreviated as PDMS) denotes, in accordance with what is generally accepted, any organosilicon polymer or oligomer of linear structure, of variable molecular weight, obtained by polymerization and/or polycondensation of suitably functionalized silanes, and consisting essentially of a repetition of main units in which the silicon atoms are linked together via oxygen atoms (siloxane bond ≡Si—O—Si≡), comprising trimethyl radicals directly linked via a carbon atom to the said silicon atoms.

The PDMS chains that may be used to obtain the copolymer used according to the invention comprise at least one polymerizable radical group, for example located on at least one of the ends of the chain, i.e. the PDMS may contain, for example, a polymerizable radical group on the two ends of the chain or one polymerizable radical group on one end of the chain and one trimethylsilyl end group on the other end of the chain. The polymerizable radical group may for example be an acrylic or methacrylic group, for example a group CH₂═CR₁—CO—O—R₂, in which R₁ represents a hydrogen or a methyl group and R₂ represents —CH₂—, —(CH₂)ₙ— with n=3, 5, 8 or 10, —CH₂—CH(CH₃)—CH₂—, —CH₂—CH₂—O—CH₂—CH₂—, —CH₂—CH₂—O—CH₂—CH₂—CH(CH₃)—CH₂—, —CH₂—CH₂CH₂—O—CH₂—CH₂—CH₂—.

The copolymers that may be used in the composition of the invention may be obtained according to the usual methods of polymerization and grafting, for example by free-radical polymerization (a) of a PDMS comprising at least one polymerizable radical group (for example on one of the ends of the chain or on both ends) and (b) of at least one carboxylic monomer, as described, for example, in documents U.S. Pat. No. 5,061,481 and U.S. Pat. No. 5,219,560.

The copolymers obtained may have a molecular weight ranging from about 3000 g/mol to about 200 000 g/mol and for example from about 5000 g/mol to about 100 000 g/mol.

A copolymer that may be used in a composition of the invention may be in its native form or in dispersed form in a solvent such as lower alcohols containing from 2 to 8 carbon atoms, for instance isopropyl alcohol, or oils, for instance volatile silicone oils (for example cyclopentasiloxane).

As copolymers that may be used in a composition of the invention, mention may be made, for example, of copolymers of acrylic acid and of stearyl acrylate containing polydimethylsiloxane grafts, copolymers of stearyl methacrylate containing polydimethylsiloxane grafts, copolymers of acrylic acid and of stearyl methacrylate containing polydimethylsiloxane grafts, copolymers of methyl methacrylate, butyl methacrylate, 2-ethylhexyl acrylate and stearyl methacrylate containing polydimethylsiloxane grafts. As a copolymer that may be used in the composition of the invention, mention may also be made of the copolymers sold by the company Shin-Etsu under the names KP-561 (CTFA name: acrylates/dimethicone), KP-541 in which the copolymer is dispersed at 60% by weight in isopropyl alcohol (CTFA name: acrylates/dimethicone and isopropyl alcohol), KP-545 in which the copolymer is dispersed at 30% in cyclopentasiloxane (CTFA name: acrylates/dimethicone and cyclopentasiloxane).

According to one exemplary embodiment of the invention, a copolymer of acrylate and of dimethicone, such as KP-561, may be used; this copolymer is not dispersed in a solvent, but is in waxy form, its melting point being about 30° C.

In the last two cases, the crystallizable side chain(s) or block(s) are hydrophobic.

The semi-crystalline polymers of the composition of the invention may or may not be partially crosslinked provided that the degree of crosslinking does not interfere with their dissolution or dispersion in the fatty phase by heating above their melting point. This may then be a case of chemical crosslinking, by reaction with a multifimctional monomer during polymerization. It may also be a case of physical crosslinidng, which may then be due either to the establishment of bonds of hydrogen or dipolar type between groups borne by the polymer, for instance dipolar interactions between carboxylate ionomers, these interactions being in small amount and borne by the polymer backbone; or to a phase separation between the crystallizable blocks and the amorphous blocks, borne by the polymer.

For example, the semi-crystalline polymers of the composition according to the invention are not crosslinked.

In practice, the total amount of semi-crystalline polymer(s) may represent from about 1% to about 8%, for example from about 2% to about 6% and for example from about 3% to about 6% by weight relative to the total weight of the composition. It may for example represent from about 4% to about 5% by weight relative to the total weight of the composition.

According to one exemplary embodiment, the ester of dimerdilinoleic acid and of polyol(s), or an ester thereof, and the semi-crystalline polymer may be present, in the compositions according to the invention, in a weight ratio that may range from about 15:1 to about 2:1, for example from about 10:1 to about 4:1 and for example from about 8:1 to about 6:1.

Deystuff

A physiologically acceptable medium of a cosmetic composition according to the invention may also comprise at least one dyestuff.

Such a dyestuff may be chosen, for example, from a water-soluble or water-insoluble, liposoluble or non-liposoluble, organic or mineral dyestuff, for example of the type such as pigments or nacres, conventionally used in cosmetic compositions.

The term “pigments” should be understood as meaning white or coloured, mineral or organic particles that are insoluble in an aqueous solution, which are intended to colour and/or opacify the resulting film.

The pigments may be present in a proportion of from 0.5% to 30% by weight, for example from 5% to 25% by weight and for example from 10% to 20% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericitelbrown iron oxide/titanium dioxide/silica type. Such a pigment is sold, for example, under the reference Coverleaf® NS or JS by the company Chemicals and Catalysts, and has a contrast ratio in the region of 30.

The dyestuff may also comprise a pigment having a structure that may be, for example, of silica microsphere type containing iron oxide. An example of a pigment having this structure is the product sold by the company Miyoshi under the reference PC BALL® PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542669, EP-A-787730, EP-A-787731 and WO-A-96/08537.

The term “nacres” should be understood as meaning coloured particles of any form, which may or may not be iridescent, for example produced by certain molluscs in their shell, or alternatively synthesized, and which have a colour effect via optical interference.

The nacres may be selected from the group constituted of nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica®, Flamenco® and Duochrome® (on mica base) sold by the company Engelhard, the Timiron® nacres sold by the company Merck, the Prestige® nacres on mica base sold by the company Eckart and the Sunshine® nacres on synthetic mica base sold by the company Sun Chemical.

The nacres may for example have a yellow, pink, red, bronze, orange, brown and/or coppery colour or glint.

As illustrations of nacres that may be used in the context of the present invention, mention may be made for example of gold-coloured nacres sold for example by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold for example by the company Merck under the names Bronze fine (17384) (Colorona) and Bronze (17353) (Colorona) and by the company Engelhard under the name Super bronze (Cloisonne); the orange nacres sold for example by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold for example by the company Engelhard under the names Nu-antique copper 340XB (Cloisonne) and Brown CL4509 (Chromalite); the nacres with a copper glint sold for example by the company Engelhard under the name Copper 340A (Timica); the nacres with a red glint sold for example by the company Merck under the name Sienna fine (17386) (Colorona); the nacres with a yellow glint sold for example by the company Engelhard under the name Yellow (4502) (Chromalite); the red-tinted nacres with a golden glint sold for example by the company Engelhard under the name Sunstone G012 (Gemtone); the pink nacres sold for example by the company Engelhard under the name Tan opale G005 (Gemtone); the black nacres with a golden glint sold for example by the company Engelhard under the name Nu antique bronze 240 AB (Timica); the blue nacres sold for example by the company Merck under the name Matte blue (17433) (Microna); the white nacres with a silvery glint sold for example by the company Merck under the name Xirona Silver; and the golden-green pinkish-orange nacres sold for example by the company Merck under the name Indian summer (Xirona), and mixtures thereof.

The cosmetic composition according to the invention may also comprise at least one water-soluble or liposoluble dye in a content ranging from 0.5% to 30% by weight and for example ranging from 5% to 25% by weight relative to the total weight of the cosmetic composition.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow. The water-soluble dyes are, for example, beetroot juice or methylene blue.

The cosmetic composition according to the invention may also contain at least one material with a specific optical effect.

This effect is different from a simple conventional hue effect, i.e. a unified and stabilized effect as produced by standard dyestuffs, for instance monochromatic pigments.

For the purposes of the invention, the term “stabilized” means lacking the effect of variability of the colour with the angle of observation or in response to a temperature change.

For example, this material may be selected from the group constituted of particles with a metallic glint, goniochromatic colouring agents, diffracting pigments, thermochromic agents, optical brighteners, and also fibres, for example interference fibres. Needless to say, these various materials may be combined so as to afford the simultaneous manifestation of two effects, or even of a novel effect in accordance with the invention.

The particles with a metallic glint that may be used in the invention may for example be selected from the group constituted of:
particles of at least one metal and/or of at least one metal derivative,
particles comprising a monomaterial or multimaterial organic or mineral substrate, at least partially coated with at least one layer with a metallic glint comprising at least one metal and/or at least one metal derivative, and
mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) may for example be used.

The term “metal derivatives” is intended to denote compounds derived from metals, for example oxides, fluorides, chlorides and sulfides.

Illustrations of these particles that may be mentioned include aluminium particles, such as those sold under the names Starbrite 1200 EAC® by the company Siberline and Metalure® by the company Eckart.

Mention may also be made of metal powders of copper or of alloy mixtures such as the references 2844 sold by the company Radium Bronze, metallic pigments, for instance aluminium or bronze, such as those sold under the names Rotosafe® 700 from the company Eckart, silica-coated aluminium particles sold under the name Visionaire Bright Silver® from the company Eckart, and metal alloy particles, for instance the silica-coated bronze (alloy of copper and zinc) powders sold under the name Visionaire Bright Natural Gold® from the company Eckart.

They may also be particles comprising a glass substrate, for instance those sold by the company Nippon Sheet Glass under the name Microglass Metashine®.

The goniochromatic colouring agent may be chosen, for example, from interference multilayer structures and liquid-crystal colouring agents.

Examples of symmetrical interference multilayer structures that may be used in compositions produced in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair® by the company Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearle by the company BASF; MoS₂/SiO₂/mica oxide/SiO₂/MoS₂; Fe₂O₃/SiO₂/mica oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of Infinite Colors® pigments from the company Shiseido. Depending on the thickness and the nature of the various coats, different effects are obtained. Thus, with the structure Fe₂O₃/SiO₂/Al/SiO₂/Fe₂O₃, the colour changes from green-golden to red-grey for SiO₂ layers of from 320 to 350 nm; from red to golden for SiO₂ layers of from 380 to 400 nm; from violet to green for SiO₂ layers of from 410 to 420 nm; from copper to red for SiO₂ layers of from 430 to 440 nm.

Examples of pigments with a polymeric multilayer structure that may be mentioned include those sold by the company 3M under the name Color Glitter®.

Examples of liquid-crystal goniochromatic particles that may be used include those sold by the company Chenix and also the product sold under the name Helicone® HC by the company Wacker.

According to one exemplary embodiment, a dyestuff that is suitable for the invention may be selected from the group constituted of organic dyestuffs and inorganic dyestuffs, such as pigments and nacres, and materials with a specific optical effect, and mixtures thereof.

According to one exemplary embodiment, a composition according to the invention may comprise not more than 30% by weight of dyestuff relative to the total weight of the composition.

Fillers

The cosmetic compositions in accordance with the invention may also comprise at least one filler, of organic or mineral nature, which for example makes it possible to give them improved stability with regard to exudation.

The term “filler” should be understood as meaning colourless or white solid particles of any form, which are in an insoluble form and dispersed in the medium of the composition. Being of mineral or organic nature they make it possible to impart body or rigidity to the composition, and/or softness, a matt effect and uniformity to the makeup.

The fillers used in the compositions according to the present invention may be of lamellar, globular or spherical form, fibres or in any other form intermediate between these defined forms.

The fillers according to the invention may or may not be surface-coated, and for instance they may be surface-treated with silicones, amino acids, fluoro derivatives or any other substance that promotes the dispersion and compatibility of the filler in the composition.

For the purposes of the present invention, the terms “mineral fillers” and “inorganic fillers” are used interchangeably.

Among the mineral fillers that may be used in the compositions according to the invention, mention may be made of talc, mica, silica, trimethyl siloxysilicate, kaolin, bentone, precipitated calcium carbonate, magnesium carbonate, magnesium hydrogen carbonate, hydroxyapatite, boron nitride, hollow silica microspheres (Silica Beads from Maprecos), glass or ceramic microcapsules, silica-based fillers, for instance Aerosil 200 or Aerosil 300; Sunsphere L-31 and Sunsphere H-31 sold by Asahi Glass; Chemicelen sold by Asahi Chemical; composites of silica and of titanium dioxide, for instance the TSG series sold by Nippon Sheet Glass, and mixtures thereof.

Among the organic fillers that may be used in the compositions according to the invention, mention may be made of polyamide powders (Nylon® Orgasol from Atochem), poly-β-alanine powder and polyethylene powder, polytetrafluoroethylene powders (Teflon®), N-lauroyl-L-lysine, starch, tetrafluoroethylene polymer powders, hollow polymer microspheres such as Expancel® (Nobel Industrie), precipitated calcium carbonate, magnesium carbonate, magnesium hydrogen carbonate, metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate, Polypore® L 200 (Chemdal Corporation), silicone resin microbeads (for example Tospearl® from Toshiba), polyurethane powders, for instance powders of crosslinked polyurethane comprising a copolymer, the said copolymer comprising trimethylol hexyllactone. For instance, it may be a polymer of hexamethylene diisocyanate/trimethylol hexyllactone. Such particles are for example commercially available, for example, under the name Plastic Powder D-400® or Plastic Powder D-800® from the company Toshiki, and mixtures thereof.

A filler may be present in a cosmetic composition in accordance with the invention in a proportion of from 0.5% to 40% by weight and for example from 5% to 30% by weight of filler relative to the total weight of the composition.

A filler that is suitable for the invention may be, for example, a filler whose mean particle size is less than 100 μm and for example between 1 and 50 μm, for example between 4 and 20 μm.

Additives

The cosmetic compositions according to the invention may, furthermore, also comprise any additive usually used in the field under consideration, selected from the group constituted of surfactants, gelling agents, filn-forming agents and, where appropriate, auxiliary film-forming agents, gums, antioxidants, essential oils, preserving agents, fragrances, neutralizers, moisturizers, antiseptics, vitamins such as vitamin B3 or E and derivatives thereof, and anti-UV agents, and mixtures thereof

The compositions according to the invention may be prepared in the usual manner for those skilled in the art. They may thus be in cast form, for example in the form of a stick or wand, in the form of a soft paste in a heating bag, or in the form of a dish that may be used by direct contact or with a sponge. For example, they may constitute, together or separately, a cast foundation, a cast makeup rouge or eyeshadow, which may be for example coloured, a lipstick, a lip gloss or a concealer product.

Each composition may be packaged separately in the same packaging article, for example in a two-compartment pen, the base composition possibly being delivered from one end of the pen and the top composition possibly being delivered from the other end of the pen, each end possibly being closed, for example in a leaktight manner, with a cap.

For example, the composition that is applied as the first coat may be in solid form, which may allow a more practical application, better stability over time and with respect to temperature of the composition, and may allow precise marking with the makeup, which may be highly desirable in the case of a lipstick or an eyeliner.

The product according to the invention may be used, for example, for making up and/or caring for the skin and/or the lips according to the nature of the ingredients used. For example, the product according to the invention may be in the form of a solid foundation, a lipstick wand or paste, a concealer product, an eye contour product, an eyeliner, a mascara, an eyeshadow, a body makeup product or a skin colouring product.

For example, the product according to the invention may be a lip makeup and/or care product, for example a lipstick.

According to an embodiment, the first and/or the second composition may be in solid form.

According to an embodiment, the lower layer applied to the skin and/or the lips may have care properties.

A subject of the invention is also a lip product, for example a lipstick, comprising a first and a second composition as described above.

The compositions according to the invention may be obtained by heating the various constituents to the melting point of the highest-melting waxes, followed by pouring the molten mixture into a mould (crucible or finger stall). They may also be obtained by extrusion, as described in patent application EP-A-0 667 146.

According to an embodiment, a subject of the present invention is also a kit comprising a product in accordance with the present invention.

A kit according to the invention may also comprise means for applying the first and/or the second composition(s) to the skin and/or the lips.

As examples of application means that are suitable for use in the present invention, mention may be made of fine brushes, coarse brushes, pens, pencils, felts, feathers, sponges and foams.

According to an embodiment, a kit in accordance with the invention may comprise a first and a second composition packaged in separate compartments or containers.

According to an embodiment, a subject of the present invention is also a process for making up and/or caring for the skin and/or the lips, comprising at least one step that consists in applying a product in accordance with the invention to at least part of a support.

According to an embodiment, a process in accordance with the invention may be performed by applying a composition comprising an ester of dimerdilinoleic acid and of polyols or an ester thereof, in accordance with the invention, as top coat.

A subject of the present invention is also the use of at least one ester of dimerdilinoleic acid and of polyols or an ester thereof, in accordance with the present invention, in combination with a physiologically acceptable medium, to prepare a first composition of a cosmetic care and/or makeup product for the skin and/or the lips, also comprising at least one second composition comprising a physiologically acceptable medium, and having improved gloss staying power.

For the purposes of the present invention, and unless otherwise indicated, the term “one” should be understood as meaning “at least one”.

The examples of compositions below are given as non-limiting illustrations of the invention.

### EXAMPLES

The examples of compositions according to the invention, presented below, of “base coat” and “top coat” type may be combined with each other to obtain a cosmetic product in accordance with the invention.

### Example 1

### Solid Base Coat

| Compound | % mass |
|---|---|
| PVP/hexadecene copolymer | 1.00 |
| Polyglyceryl-2 triisostearate | 1.00 |
| Isononyl isononanoate | 1.00 |
| Neopentyl glycol dicaprate | 1.00 |
| Phenyl trimethicone | 1.00 |
| Hydrogenated polyisobutene | 4.00 |
| Isononyl isononanoate | 3.00 |
| Neopentyl glycol dicaprate | 3.00 |
| Phenyl trimethicone | 3.00 |
| Isododecane | 2.00 |
| D5 | 38.00 |
| PSPA | 4.00 |
| MQ Resin | 6.00 |
| BHT | 0.05 |
| Polyethylene (M = 500) | 14.29 |
| Candelilla wax | 1.90 |
| Titanium dioxide | 0.19 |
| D&C Red 7 W 012 C | 0.43 |
| Iron oxides | 0.90 |
| Yellow 5 lake | 0.81 |
| Blue 1 lake | 0.19 |
| Ethylene glycol dimethacrylate/lauryl | 0.95 |
| methacrylate copolymer | |
| N-Lauroyl-L-lysine | 0.95 |
| Quaternium-18 hectorite | 2.05 |
| Silica | 5.00 |
| Mica-coated iron oxide | 2.67 |
| Calcium aluminium borosilicate-coated | 0.48 |
| titanium dioxide | |
| Mica-coated titanium dioxide | 0.95 |
| Simethicone | 0.19 |
| TOTAL | 100.00 |

Procedure

An oily phase is prepared by mixing the oils together, with heating (about 95° C.).

The oily phase thus prepared is stirred at about 95° C. and the fillers are added to the mixture.

The waxes, the pigments in the form of a pigmentary paste, prepared by mixing the dyestuffs with the polyglyceryl triisostearate and the isononyl isononanoate, and the simethicone are then added to the mixture.

### Example 2

### Liquid Base Coat

| Compound | % mass |
|---|---|
| PVP/hexadecene copolymer | 1.00 |
| Polyglyceryl-2 triisostearate | 1.00 |
| Isononyl isononanoate | 1.00 |
| Neopentyl glycol dicaprate | 1.00 |
| Phenyl trimethicone | 1.00 |
| Hydrogenated polyisobutene | 4.00 |
| Isononyl isononanoate | 3.00 |
| Neopentyl glycol dicaprate | 3.00 |
| Phenyl trimethicone | 3.00 |
| Isododecane | 2.00 |
| D5 | 38.00 |
| PSPA | 4.00 |
| MQ Resin | 6.00 |
| BHT | 0.05 |
| Microcrystalline wax | 5.00 |
| Titanium dioxide | 0.19 |
| D&C Red 7 W 012 C | 0.43 |
| Iron oxides | 0.90 |
| Yellow 5 lake | 0.81 |
| Blue 1 lake | 0.19 |
| Ethylene glycol dimethacrylate/lauryl | 0.95 |
| methacrylate copolymer | |
| N-Lauroyl-L-lysine | 0.95 |
| Quaternium-18 hectorite | 11.24 |
| Silica | 7.00 |
| Mica-coated iron oxide | 2.67 |
| Calcium aluminium borosilicate-coated | 0.48 |
| titanium dioxide | |
| Mica-coated titanium dioxide | 0.95 |
| Simethicone | 0.19 |
| TOTAL | 100.00 |

The composition was prepared as indicated in Example 1.

### Example 3

### Top Coat

| Compound | % mass |
|---|---|
| Polyethylene (Performalene 500 polyethylene - | 2.00 |
| New Phase Technologies) | |
| Ozokerite | 3.00 |
| Beeswax | 3.00 |
| Microcrystalline wax | 2.00 |
| Petroleum wax (EMW-3 (Nippon Seiro)) | 8.00 |
| Hydrogenated polyisobutene | 10.00 |
| Diisostearyl malate | 10.00 |
| Neopentyl glycol dicaprate | 10.00 |
| Isotridecyl isononanoate | 10.00 |
| Diisostearyl malate | 8.00 |
| Polyglyceryl-2 triisostearate (Salacos 43N | 5.00 |
| (Nisshin Oillio)) | |
| Polyglyceryl-2 isostearate dimerdilinoleate | 10.00 |
| copolymer (Hailucent ISDA) | |
| Dimerdilinoleyl dimerdilinoleate (Lusplan | 2.00 |
| DD-DA7 (NFC)) | |
| Bis-behenyl/isostearyl/phytosteryl | 5.00 |
| dimerdilinoleyl dimerdilinoleate (Plandool-G | |
| (Nippon Fine Chemicals)) | |
| Acrylate/stearyl acrylate/dimethicone | 2.00 |
| methacrylate copolymer | |
| Trimethyl siloxysilicate | 2.00 |
| Dimethicone | 0.90 |
| Titanium-coated mica | 5.00 |
| Calcium aluminium borosilicate (Metashine: | 2.00 |
| Nippon Sheet Glass) | |
| D&C Red 28 | 0.10 |
| TOTAL | 100.00 |

An oily phase is prepared by mixing together with heating (about 95° C.) the oils and the polyglyceryl-2 isostearate dimerdilinoleate copolymer (Hailucent ISDA).

The oily phase thus prepared is stirred at about 95° C. and the fillers are added to the mixture.

The waxes, the pigments in the form of a pigmentary paste, prepared by mixing the dyestuffs with the polyglyceryl-2 triisostearate and the isononyl isononanoate and the simethicone, the bis-behenyl/isostearyl/phytosteryl dimerdilinoleyl dimerdilinoleate (Plandool-G) and the dimerdilinoleyl dimerdilinoleate are then added to the mixture.

### Example 4

### Top Coat

| Compound | % mass |
|---|---|
| Polyethylene (Performalene 500 polyethylene - | 2.00 |
| New Phase Technologies) | |
| Ozokerite | 3.00 |
| Beeswax | 3.00 |
| Microcrystalline wax | 2.00 |
| Petroleum wax (EMW-3 (Nippon Seiro)) | 6.00 |
| Hydrogenated polyisobutene | 10.00 |
| Diisostearyl malate | 10.00 |
| Neopentyl glycol dicaprate | 10.00 |
| Isotridecyl isononanoate | 10.00 |
| Polyglyceryl-2 triisostearate (Salacos 43N | 5.00 |
| (Nisshin Oillio)) | |
| Polyglyceryl-3 polydimethyl siloxyethyl | 5.00 |
| dimethicone (KF6104 (Shin-Etsu)) | |
| Dimethicone copolyol (KF6017 (Shin-Etsu)) | 5.00 |
| Polyglyceryl-2 isostearate/dimerdilinoleate | 10.00 |
| copolymer (Hailucent ISDA) | |
| Dimerdilinoleyl dimerdilinoleate (Lusplan | 2.00 |
| DD-DA7 (NFC)) | |
| Bis-behenyl/isostearyl/phytosteryl | 5.00 |
| dimerdilinoleyl dimerdilinoleate (Plandool-G | |
| (Nippon Fine Chemicals)) | |
| Acrylate/stearyl acrylate/dimethicone | 2.00 |
| methacrylate copolymer | |
| Trimethyl siloxysilicate | 2.00 |
| Dimethicone | 0.90 |
| Titanium-coated mica | 5.00 |
| Calcium aluminium borosilicate (Metashine: | 2.00 |
| Nippon Sheet Glass) | |
| D&C Red 28 | 0.10 |
| TOTAL | 100.00 |

The composition was prepared as indicated in Example 3.

### Example 5

### Top Coat

| Compound | % mass |
|---|---|
| Polyethylene (Performalene 500 polyethylene - | 2.00 |
| New Phase Technologies) | |
| Ozokerite | 3.00 |
| Beeswax | 3.00 |
| Microcrystalline wax | 2.00 |
| Petroleum wax (EMW-3 (Nippon Seiro)) | 6.00 |
| Sucrose polycottonseedate | 2.00 |
| Hydrogenated polyisobutene | 10.00 |
| Diisostearyl malate | 10.00 |
| Neopentyl glycol dicaprate | 10.00 |
| Isotridecyl isononanoate | 10.00 |
| Diisostearyl malate | 8.00 |
| Polyglyceryl-2 triisostearate (Salacos 43N | 5.00 |
| (Nisshin Oillio)) | |
| Polyglyceryl-3 isostearate/dimerdilinoleate | 10.00 |
| Copolymer (Hailucent ISDA) | |
| Dimerdilinoleyl dimerdilinoleate (Lusplan | 2.00 |
| DD-DA7 (NFC)) | |
| Bis-behenyl/isostearyl/phytosteryl | 5.00 |
| dimerdilinoleyl dimerdilinoleate (Plandool-G | |
| (Nippon Fine Chemicals)) | |
| Acrylate/stearyl acrylate/dimethicone | 2.00 |
| methacrylate copolymer | |
| Trimethyl siloxysilicate | 2.00 |
| Dimethicone | 0.90 |
| Titanium-coated mica | 5.00 |
| Calcium aluminium borosilicate (Metashine: | 2.00 |
| Nippon Sheet Glass) | |
| D&C Red 28 | 0.10 |
| TOTAL | 100.00 |

The composition was prepared as indicated in Example 3.

1. Cosmetic product for caring for and/or making up the skin and/or the lips, comprising at least:
one first composition comprising, in a physiologically acceptable medium, at least one ester of dimerdilinoleic acid and of polyol(s) or an ester thereof, the viscosity of which, measured at about 25° C., is greater than or equal to about 1500 mpa·s, and
one second composition comprising a physiologically acceptable medium.

2. Product according to claim 1, in which the said ester has a molecular weight ranging from about 2000 to about 25 000 g/mol.

3. Product according to claim 1, in which the polyol is a diol.

4. Product according to claim 3, in which the said ester comprises an alternating sequence of dimerdilinoleate residue(s) and of residue(s) related to the said diol(s).

5. Product according to claim 3, in which the diol is selected from the group constituted of a fatty alcohol dimer, a monoglycerol or polyglyceryl, a C₂₋₄ monoalkylene or polyalkylene glycol, 1,4-butanediol and pentaerythritol.

6. Product according to claim 5, in which the fatty alcohol dimer is the product of hydrogenation of a fatty acid dimer obtained by dimerization of a C₈ to C₃₄ unsaturated fatty acid.

7. Product according to claim 4, in which each of the two ends of the said sequence bears, respectively, a unit OR′ and OR″ with R′ and R″ representing, independently of each other, a hydrogen atom or OR′ and OR″, representing, independently of each other, a C₂ to C₃₆ hydrocarbon-based monoalcohol residue.

8. Product according to claim 7, in which R′ and R″ both represent a hydrogen atom.

9. Product according to claim 7, in which OR′ and OR″ both represent an identical or different hydrocarbon-based monoalcohol residue.

10. Product according to claim 1, comprising an ester of dimerdilinoleic acid and of polyol(s) or an ester thereof, the viscosity of which, measured at about 25° C., is greater than or equal to about 2000 mPa·s.

11. Product according to claim 1, comprising an ester of general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue,
OR₃ represents a hydrocarbon-based monoalcohol residue, and
n is an integer ranging from 1 to 15.

12. Product according to claim 1, comprising of general formula (II) below: 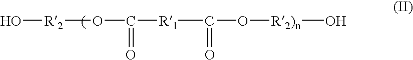
in which:
n is an integer ranging from 1 to 15,
—OCR′₁CO represents a dimerdilinoleate residue,
OR′₂O represents a diglyceryl residue of general formula (III) below: 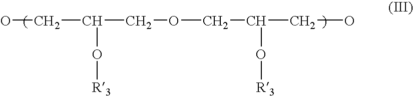
in which:
R′₃ represents H or OR′₃ represents a fatty acid residue.

13. Product according to claim 1, comprising an ester of general formula (IV):
HO—R₁″—(—OCO—R₂″—COO—R₁″—)ₕ—OH (IV)
in which:
OR₁″O represents a diol dimer residue obtained by hydrogenation of a dimerdilinoleic acid,
COR₂″CO represents a hydrogenated dimerdilinoleate residue, and
h represents an integer ranging from 1 to 9.

14. Product according to claim 1, in which the said ester is selected from the group constituted of the esters of the following INCI nomenclature: polyglyceryl-2 sostearate/dimerdilinoleate copolymer, bis-behenyl/isostearyl/phytosteryl dimerdilinoleyl imerdilinoleate, dimerdilinoleyl dimerdilinoleate and mixtures thereof.

15. Product according to claim 1, wherein the composition(s) comprise(s) from 5% to 90% by weight of the said ester relative to the total weight of the composition.

16. Product according to claim 1, in which the physiologically acceptable medium also comprises at least one liquid fatty phase selected from the group constituted of volatile oils and non-volatile oils, and mixtures thereof.

17. Product according to claim 16, in which the volatile oil is selected from the group constituted of:
hydrocarbon-based oils containing from 8 to 16 carbon atoms,
linear or cyclic silicone oils,
volatile fluoro oils, and
mixtures thereof.

18. Product according to claim 16, in which the non-volatile oil is an oil of molar mass ranging from about 650 to about 10 000 g/mol.

19. Product according to claim 18, in which the said oil is selected from the group constituted of:
lipophilic polymers,
linear fatty acid esters with a total carbon number ranging from 35 to 70,
hydroxylated esters,
aromatic esters,
esters of C₂₄-C₂₈ branched fatty alcohols or fatty acids,
silicone oils,
oils of plant origin,
and mixtures thereof.

20. Product according to claim 18, in which the said oil is selected from the group constituted of polybutylenes, hydrogenated polyisobutylenes, polydecenes, hydrogenated polydecenes, vinylpyrrolidone copolymers, pentaerythrityl tetrapelargonate, polyglyceryl-2 triisostearate, tridecyl trimellitate, triisoarachidyl citrate, pentaerythrityl tetraisononanoate, glyceryl triisostearate, glyceryl tris(2-decyl)tetradecanoate, pentaerythrityl tetraisostearate, polyglyceryl-2 tetraisostearate, pentaerythrityl tetrakis(2-decyl)tetradecanoate, phenyl silicones, sesame oil and oils of an ester of an acid dimer and of alcohol, and mixtures thereof.

21. Product according to claim 1, in which the physiologically acceptable medium also comprises at least one solid fatty phase selected from the group constituted of pasty fatty substances and waxes, and mixtures thereof.

22. Product according to claim 1, in which the physiologically acceptable medium also comprises at least one dyestuff.

23. Product according to claim 22, in which the dyestuff is selected from the group constituted of organic dyestuffs, inorganic dyestuffs, and materials with a specific optical effect, and mixtures thereof.

24. Product according to claim 1, in which the physiologically acceptable medium also comprises a semi-crystalline polymer.

25. Product according to claim 24, in which the said semi-crystalline polymer has a melting point of less than about 50° C.

26. Product according to claim 24, in which the said semi-crystalline polymer is a polymer comprising a) a hydrocarbon-based or silicone polymer backbone and b) at least one crystallizable organic side chain and/or one crystallizable organic block forming part of the polymer backbone of the said polymer, the said polymer having a number-average molecular mass of greater than or equal to about 2000 g/mol.

27. Product according to claim 24, in which the said semi-crystalline polymer is selected from the group constituted of copolymers resulting from the polymerization (a) of one or more carboxylic monomer(s) with (b) one or more polydimethylsiloxane chain(s) comprising at least one polymerizable radical.

28. Product according to claim 27, in which the monomer is selected from the group constituted of acrylic acid, methacrylic acid, maleic acid, fumaric acid, itaconic acid, crotonic acid, and esters thereof, and mixtures thereof.

29. Product according to claim 24, in which the semi-crystalline polymer is a copolymer of acrylate and of dimethicone.

30. Product according to claim 1, in which at least one of the two compositions is in the form of a suspension, a dispersion, a solution, a gel, an emulsion, or in the form of a cream, a stick, a paste, a mousse, a vesicular dispersion, a two-phase or multi-phase lotion or a powder.

31. Product according to claim 1, in which at least one of the two compositions is anhydrous.

32. Product according to claim 1, which is a lip makeup and/or care product.

33. Cosmetic product for caring for and/or making up the skin and/or the lips, comprising at least two different compositions and containing, respectively, at least one ester of dimerdilinoleic acid and of polyol(s) or an ester thereof, the viscosity of which, measured at about 25° C., is greater than or equal to about 2000 mPa·s.

34. Product according to claim 33, in which the esters contained in each of the two compositions are different.

35. Cosmetic care and/or makeup product for the skin and/or the lips, comprising at least two different compositions with at least one composition containing at least one ester of general formula (I) below:
R₃—OCO—R₁(—COO—R₂—OCO—R₁)ₙ—COO—R₃ (I)
in which:
OCR₁CO represents a dimerdilinoleate residue,
OR₂O represents a fatty alcohol dimer residue,
OR₃ represents a hydrocarbon-based monoalcohol residue, and
n is an integer ranging from 1 to 15.

36. Product according to claim 35, in which OR₂O represents a dimerdilinoleyl residue.

37. Product according to claim 35, in which OR₃ represents a hydrocarbon-based monoalcohol residue selected from the group constituted of behenyl, isostearyl and phytosteryl residues, and mixtures thereof.

38. Cosmetic product for caring for and/or making up the skin and/or the lips, comprising at least two different compositions, with at least one composition containing at least one ester of general formula (II) below: 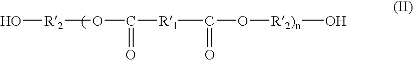
in which:
n is an integer ranging from 1 to 15,
—OCR′₁CO represents a dimerdilinoleate residue,
OR′₂O represents a diglyceryl residue of general formula (III) below: 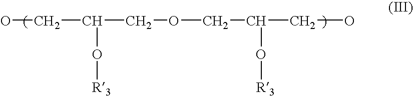
in which:
R′₃ represents H or OR′₃ represents a fatty acid residue.

39. Product according to claim 38, in which the fatty acid residue featured by OR′₃ is an isostearyl residue.

40. Cosmetic product for caring for and/or making up the skin and/or the lips, comprising at least two different compositions, with at least one composition containing at least one ester of general formula (IV):
HO—R₁″—(—OCO—R₂″—COO—R₁″—)ₕ—OH (IV)
in which:
OR₁″O represents a diol dimer residue obtained by hydrogenation of a dimerdilinoleic acid,
COR₂″CO represents a hydrogenated dimerdilinoleate residue, and
h represents an integer ranging from 1 to 9.

41. Makeup kit comprising at least one product as defined in claim 1.

42. Kit according to claim 41, containing means for applying the first and/or the second composition(s) to the skin and/or the lips.

43. Kit according to claim 41, containing application means selected from the group constituted of fine brushes, coarse brushes, pens, pencils, felts, fibres, sponges and foams.

44. Kit according to claim 41, wherein the first and second compositions are packaged in separate compartments or containers.

45. Process for making up and/or caring for the skin and/or the lips, comprising at least one step that consists in applying, to at least part of a support, a product as defined in claim 1.

46. Process according to claim 45, wherein the composition containing the said ester is applied as a top coat.
